(19) United States
(12) Patent Application Publication
(10) Pub. No.: US 2012/0302850 A1
(43) Pub. Date: Nov. 29, 2012

(54) METHOD AND APPARATUS FOR MEASURING A HUMAN PHYSIOLOGICAL SIGNAL

(75) Inventors: Shu-Hung LIN, Taipei Hsien (TW); Chia-Hsien Li, Taipei Hsien (TW); Yao-Tsung Chang, Taipei Hsien (TW); Shun-Chi Chung, Taipei Hsien (TW); Pai-Yang Lin, Taipei Hsien (TW)

(73) Assignee: Wistron Corporation, Taipei Hsien (TW)

(21) Appl. No.: 13/414,833

(22) Filed: Mar. 8, 2012

(30) Foreign Application Priority Data
May 27, 2011 (TW) 100118695

(51) Int. Cl. A61B 5/00; A61B 5/0402; A61B 5/145; A61B 5/021

(52) U.S. Cl. 600/365; 600/300; 600/485; 600/509

(57) ABSTRACT

A method for measuring a human physiological signal includes: receiving a reminder canceling time range; measuring a human physiological signal by a human physiological signal measuring apparatus; and determining if a measurement-related time for measuring the physiological signal falls within the reminder canceling time range, followed by releasing a reminding device in response to an affirmative determination.

### BACKGROUND OF THE INVENTION

1. Field of the Invention

The present invention relates to a method for measuring a human physiological signal, and more particularly, to a method for measuring a human physiological signal to remind a user according to whether a measurement-related time falls within a reminder canceling time range.

2. Description of the Prior Art

In modern society, due to medical advancements, people today are attaching increasingly greater importance to personal health and thus often need to measure human physiological signals related to blood pressure, blood sugar, body fat, etc. However, people often forget to undertake the aforesaid measurement tasks for various reasons, such as being busy working. At present, related commercially available apparatuses are equipped mostly with a reminding device for reminding a user to undertake measurement tasks. The reminding devices each usually comprise a mechanism of an alarm clock for giving a reminder to the user as soon as a preset time period expires. However, measurement of a human physiological signal does not necessarily take place at a specific point in time. Hence, the aforesaid way of setting is likely to cause annoyance to the user and thus requires improvement.

Accordingly, the present invention provides a method and apparatus for measuring a human physiological signal with a view to overcoming the drawbacks of the prior art by setting a reminder time range based on a typical user's habits.

### SUMMARY OF THE INVENTION

It is a primary objective of the present invention to provide a method for measuring a human physiological signal, the method being for use with an apparatus for measuring a human physiological signal, the apparatus comprising a reminding device.

Another primary objective of the present invention is to provide an apparatus for measuring a human physiological signal by allowing a user to measure a human physiological signal.

In order to achieve the above and other objectives, the present invention provides a method for measuring a human physiological signal. The method comprises the steps of: receiving a reminder canceling time range; measuring a physiological signal with the human physiological signal measuring apparatus; and determining whether a measurement-related time for measuring the physiological signal falls within the reminder canceling time range, followed by releasing the reminding device in response to an affirmative determination.

The apparatus for measuring a human physiological signal according to the present invention comprises a receiving module, a measuring module, a reminding device, a clock device, a controlling module, and a memory. The receiving module receives a reminder canceling time range. The measuring module measures a physiological signal. The reminding device reminds a user to measure a physiological signal. The clock device provides a measurement-related time for measuring a physiological signal. The memory stores the reminder canceling time range. The controlling module is electrically connected with and adapted to control the receiving module, the measuring module, the reminding device, the clock device, and the memory for determining whether a measurement-related time falls within a reminder canceling time range and releasing the reminding device in response to an affirmative determination.

### BRIEF DESCRIPTION OF THE DRAWINGS

FIGS. 1(A), 1(B), and 1(C) are schematic views of an environment where an apparatus for measuring a human physiological signal operates according to the present invention;

FIG. 2 is a schematic view of the apparatus for measuring a human physiological signal according to the present invention;

FIG. 3 is a flowchart of setting a reminder canceling time range according to the present invention;

FIG. 4 is a flowchart of determining whether to release a reminding device according to the present invention;

FIG. 5 is a flowchart of recording and transmitting related numerical data according to the present invention; and

FIG. 6 is a table of set times in a specific embodiment according to the present invention.

### DETAILED DESCRIPTION OF THE EMBODIMENTS

To enable persons skilled in the art to gain insight into the technical contents of the present invention, the present invention is hereunder illustrated with preferred embodiments and accompanying drawings.

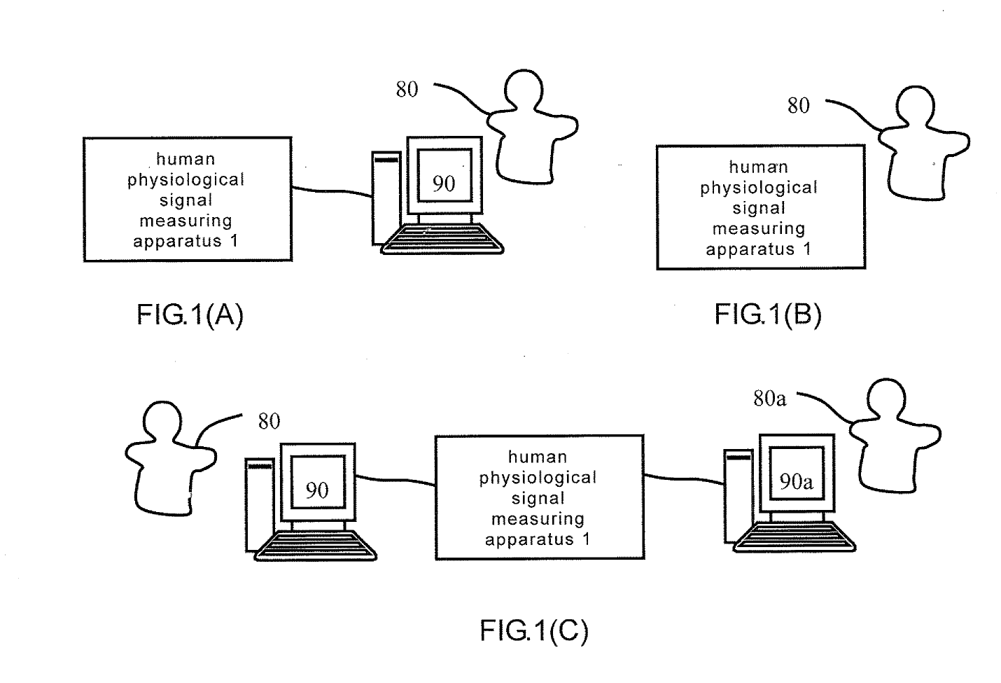

Please referring to FIG. 1(A), FIG. 1(B) and FIG. 1(C), in which are shown schematic views of an environment where an apparatus for measuring a human physiological signal operates according to the present invention. As shown in FIG. 1(A), a human physiological signal measuring apparatus 1 of the present invention is connected to an external electronic device 90 for allowing a user 80 to set a time for measuring a physiological signal and to set a reminding device by means of the external electronic device 90. Upon completion of the setting process, the user 80 can use the human physiological signal measuring apparatus 1 to measure a physiological signal. In an embodiment of the present invention, although the external electronic device 90 is a personal computer, the disclosure of the present invention is not limited thereto; instead, it is feasible to set the time by means of a PDA or a smart phone.

The application of the human physiological signal measuring apparatus 1 of the present invention is not limited to being connected with the external electronic device 90. Referring to FIG. 1(B), the user 80 can also set the human physiological signal measuring apparatus 1 of the present invention directly, without involving the external electronic device 90. Referring to FIG. 1(C), the application of the human physiological signal measuring apparatus 1 is not limited to one person only; instead, it is also feasible for multiple different users; such as the user 80 and a user 80a, to operate the external electronic device 90 and an external electronic device 90a, respectively, for setting different measurement times as needed.

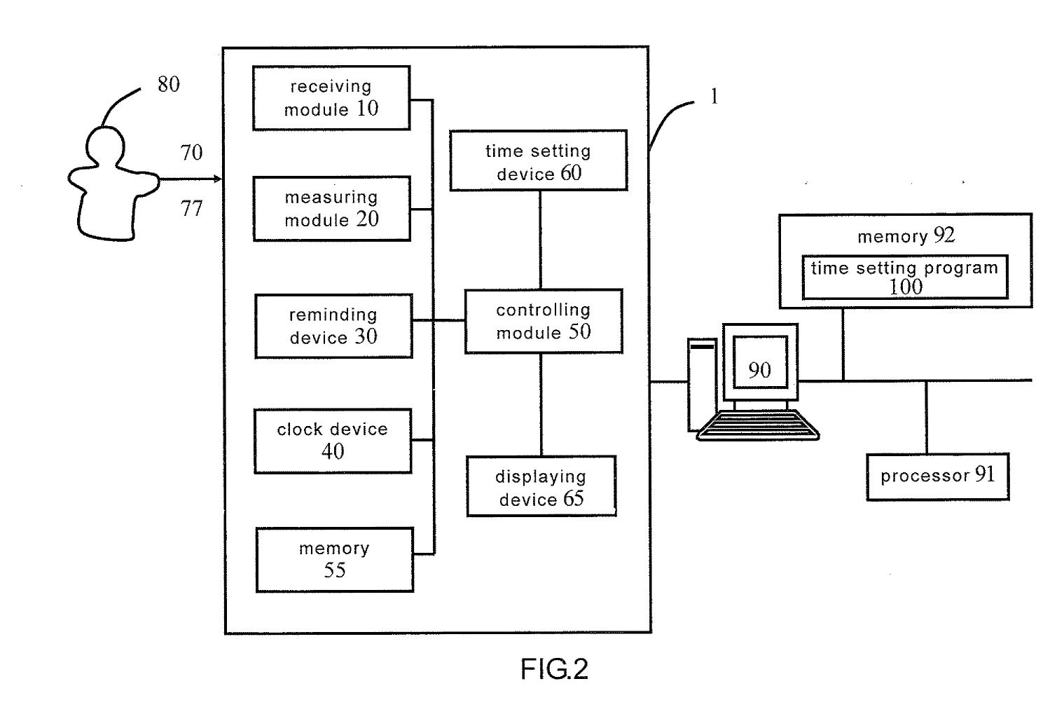

Please refer to FIG. 2, in which is shown a schematic view of the apparatus for measuring a human physiological signal according to the present invention. The human physiological signal measuring apparatus 1 of the present invention is connected to the external electronic device 90 and comprises a receiving module 10, a measuring module 20, a reminding device 30, a clock device 40, a controlling module 50, a memory 55, a time setting device 60, and a displaying device 65. In an embodiment of the present invention, the aforesaid elements can not only come in the form of a hardware device, a software program, firmware, or a combination thereof, but also come in the form of a circuit loop or any other appropriate arrangement. If any one of the elements is implemented by software, it can be loaded on a computer-readable storage medium. Each of the elements can be self-contained; alternatively, the elements can operate in conjunction with each other. Furthermore, this embodiment is intended to be illustrative of a preferred embodiment of the present invention, and is not described in detail in terms of any possible combination of variations for the sake of brevity. However, persons skilled in the art should be able to understand that the present invention can, for the purpose of the implementation thereof, require any other more delicate conventional modules or elements. The modules or elements can be omitted or altered as needed. Any other module or element is not necessarily absent between any two of the modules.

In an embodiment of the present invention, the external electronic device 90 comprises a processor 91 and a memory 92. A time setting program 100 is stored in the memory 92. The user 80 operates the external electronic device 90 and executes the time setting program 100 in the memory 92 by means of the processor 91 to set the human physiological signal measuring apparatus 1. The receiving module 10 receives a reminder canceling time range. The measuring module 20 measures a physiological signal 70. The reminding device 30 reminds the user 80 to undertake a measurement task. The clock device 40 provides a measurement-related time for measuring the physiological signal 70. The memory 55 stores the reminder canceling time range. The controlling module 50 is electrically connected with, and adapted to control, the receiving module 10, the measuring module 20, the reminding device 30, the clock device 40, and the memory 55 for determining whether the measurement-related time falls within the reminder canceling time range and releasing the reminding device 30 in response to an affirmative determination.

In an embodiment of the present invention, the measurement-related time is the time taken to measure a physiological signal, and the reminder canceling time range is 15 minutes to 90 minutes, though the disclosure of the present invention is not limited thereto. The human physiological signal measuring apparatus 1 further comprises the time setting device 60 for setting a preset reminder starting time or the reminder canceling time range and the displaying device 65 for displaying a measurement record. The time setting device 60 and the displaying device 65 are not indispensible to the present invention. In an embodiment of the present invention, an external electronic device sets the preset reminder starting time and the reminder canceling time range and displays a measurement record. Furthermore, the receiving module 10 further receives the preset reminder starting time and obtains the reminder canceling time range based on the preset reminder starting time, wherein the preset reminder starting time falls within the reminder canceling time range. The receiving module 10 further receives an identification data 77 and identifies a user identity based on the identification data 77. The reminding device 30 is an audio reminding device for generating sound to remind the user 80 to undertake a measurement task, though the disclosure of the present invention is not limited thereto. Alternatively, the reminding device 30 is a light-emitting reminding device or any other type of reminding device. The physiological signal 70 is a blood pressure-related signal, an electrocardiogram-related signal, or a blood sugar-related signal. Likewise, the measuring module 20 is a blood pressure measuring module, an electrocardiogram measuring module, or a blood sugar measuring module, though the disclosure of the present invention is not limited thereto.

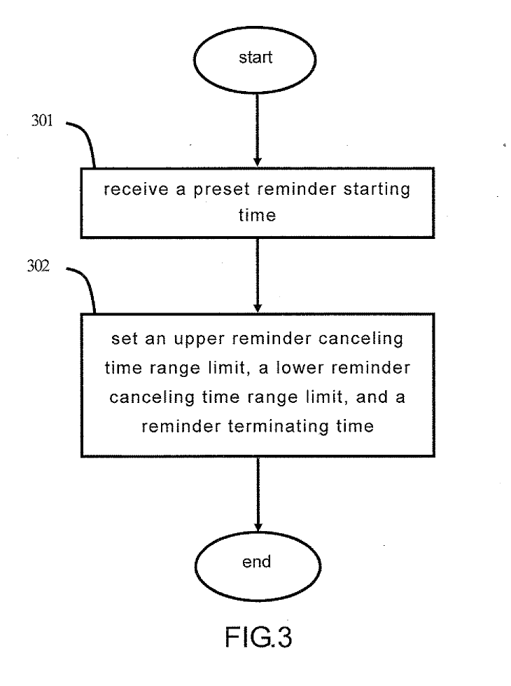

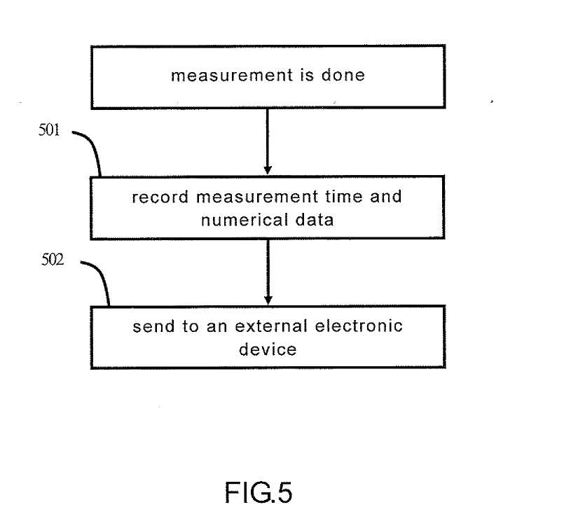

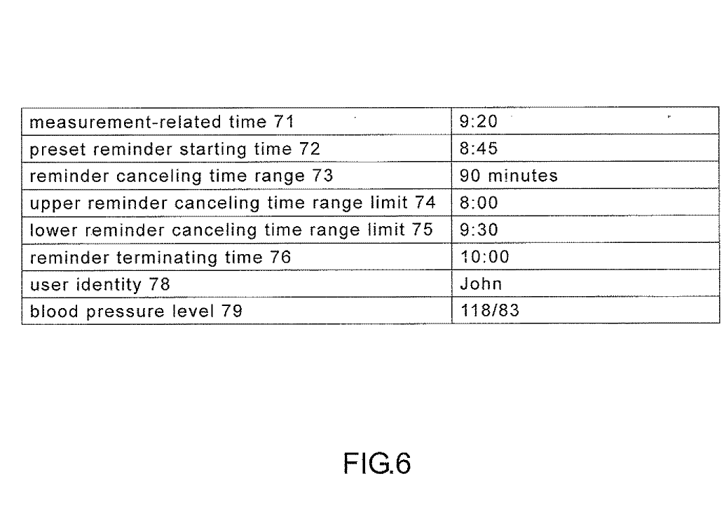

Please refer to FIG. 3 through FIG. 5, in which are shown the flowcharts of a method for measuring a human physiological signal according to the present invention. Also, please refer to FIG. 2 and FIG. 6, in which are shown diagrams of a specific embodiment of the present invention. Although the method for measuring a human physiological signal according to the present invention is illustrated with the human physiological signal measuring apparatus 1 shown in FIG. 2, the method of the present invention is not limited to application of the human physiological signal measuring apparatus 1.

Step 301: receive a preset reminder starting time.

Please refer to FIG. 6, in which is shown a table of set times in a specific embodiment according to the present invention. The user 80 operates the external electronic device 96 and executes the time setting program 100 in the memory 92 by means of the processor 91 to set a preset reminder starting time 72. Afterward, the preset reminder starting time 72 is received by the human physiological signal measuring apparatus 1 of the present invention and stored in the memory 55. In this embodiment, the user 80 sets the preset reminder starting time 72 to 8:45 AM.

Step 302: set an upper reminder canceling time range limit, a lower reminder canceling time range limit, and a reminder terminating tame.

Afterward, the user 80 sets a reminder canceling time range by executing the time setting program 100. Referring to FIG. 6, the user 80 sets a reminder canceling time range 73 to 8:00 AM-9:30 AM, wherein the user 80 sets an upper reminder canceling time range limit 74 to 8:00 AM and a lower reminder canceling time range limit 75 to 9:30 AM. Also, the user 80 can set a reminder terminating time 76 to 10:00 AM. In this embodiment, the reminder canceling time range 73 is 90 minutes, though the disclosure of the present invention is not limited thereto. In this embodiment, the user 80 sets the reminder canceling time range 73 and the reminder terminating time 76 directly; however, the reminder canceling time range 73 and the reminder terminating time 76 can also be automatically obtained by the receiving module 10 in accordance with the preset reminder starting time 72, not to mention that the preset reminder starting time 72 falls within the reminder canceling time range 73.

The aforesaid step 301 and step 302 describe how the user 80 operates the external electronic device 90 and executes the time setting program 100 in the memory 92 by means of the processor 91 to set the preset reminder starting time 72. The method for measuring a human physiological signal according to the present invention is not necessarily executed by the external electronic device 90; instead, the user 80 can also directly use the time setting device 60 (the time is usually set by means of a hardware button or a software button, either of which operates in conjunction with the controlling module 50; with time setting being a prior art, it is not described in detail herein for the sake of brevity) to perform a setting operation, without using the external electronic device 90.

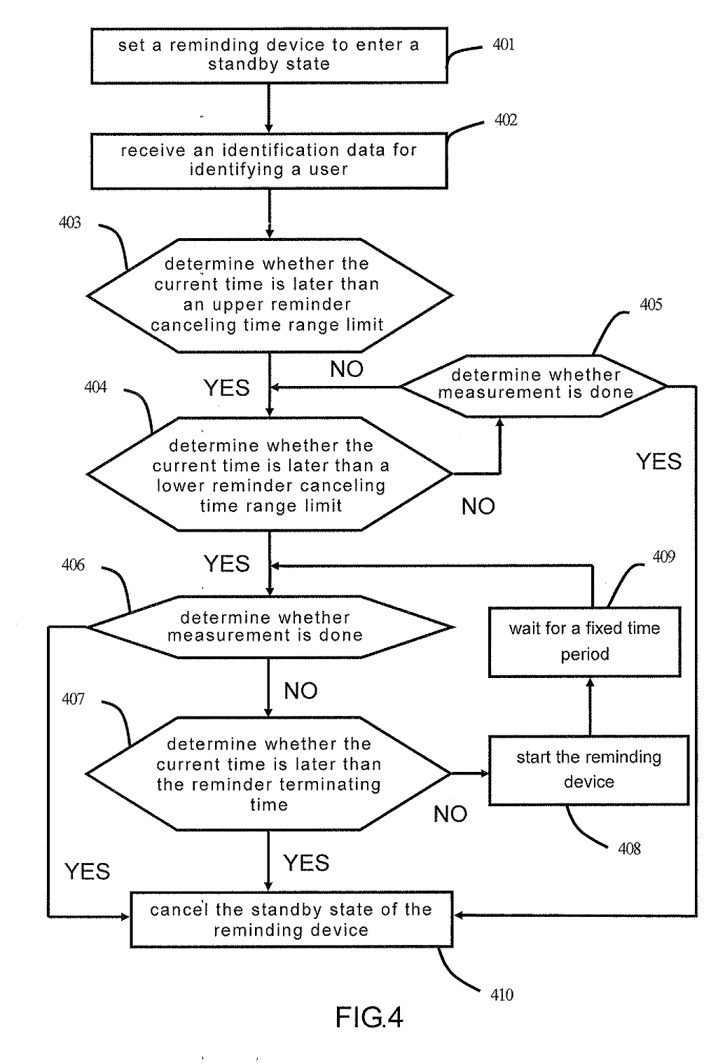

Please refer to FIG. 4, in which is shown a flowchart of determining whether to release a reminding device according to the present invention. Upon completion of setting the upper reminder canceling time range limit 74, the lower reminder canceling time range limit 75, and the reminder terminating time 76, the steps below are performed.

Step 401: set a reminding device to enter a standby state.

The reminding device 30 enters a standby state. Once the reminding device 30 enters the standby state, the reminding device 30 can start at any time. In this embodiment, the reminding device 30 is an audio reminding device, though the disclosure of the present invention is not limited thereto.

Step 402: receive an identification data for identifying a user.

Afterward, the user 80 sends the identification data 77 to the human physiological signal measuring apparatus 1 by means of the electronic device 90 or the human physiological signal measuring apparatus 1. Then, the identification data 77 is received by the receiving module 10 for identifying an identity of the user 80 based on the identification data 77. Referring to FIG. 6, in this embodiment, a user identity of the user 80 is identified as “John” by the receiving module 10 according to the identification data 77. Since a way of identifying a user's identity is a prior art, it is not described in detail herein for the sake of brevity. In a multiple-user mode (see FIG. 1(C)), users send their respective said identification data 77 to the human physiological signal measuring apparatus 1 by means of electronic devices, respectively, and then the identification data 77 is identified before a measurement task begins.

Step 403; determine whether the current time is later than an upper reminder canceling time range limit.

As time passes, the controlling module 50 determines whether the current time is later than the upper reminder canceling time range limit 74. If it is determined that the current time is later than the upper reminder canceling time range limit 74, step 404 will begin. In this embodiment, the controlling module 50 knows the current time through the clock device 40 and determines whether the current time is later than 8:00 AM. If it is determined that the current time is later than 8:00 AM, step 404 will begin.

If it is determined that the current time is later than the upper reminder canceling time range limit, the process will go to step 404: determine whether the current time is later than the lower reminder canceling time range limit.

If it is determined that the current time is later than the upper reminder canceling time range limit 74, the controlling module 50 will further determine whether the current time is later than the lower reminder canceling time range limit 75. If it is determined that the current time is later than the lower reminder canceling time range limit 75, step 406 will begin; otherwise, step 405 will begin. In this embodiment, the controlling module 50 determines whether the current time is later than 9:30 AM. If it is determined that the current time is later than 9:30 AM, step 406 will begin; otherwise, step 405 will begin.

If it is determined that the current time is not later than the lower reminder canceling time range limit, go to step 405: determine whether the measurement is done.

If it is determined that the current time is not later than the lower reminder canceling time range limit 75, the controlling module 50 will further determine whether the measurement is done. If it is determined that the measurement is done, then the process will go to step 410 for canceling the standby state of the reminding device 30; otherwise, the process will go back to step 404. In this embodiment, the controlling module 50 is connected to the measuring module 20 for determining whether the measurement is done. If it is determined that the measurement is done before 9:30 AM, that is, the lower reminder canceling time range limit 75, the standby state of the reminding device 30 will be canceled; otherwise, the process will go back to step 404.

If it is determined that the current time is later than the lower reminder canceling time range limit, then the process will go to step 406: determine whether the measurement is done.

If it is determined that the current time is later than the lower reminder canceling time range limit 75, the controlling module 50 will determine whether the measurement is done. If it is determined that the measurement is done, step 410 will begin, thereby canceling the standby state of the reminding device 30; otherwise, step 407 will begin. In this embodiment, the controlling module 50 is connected to the measuring module 20 for determining whether the measurement is done. If it is determined that the measurement is done, the standby state of the reminding device 30 will be canceled; otherwise, the process will go to step 407.

If the measurement is not done, then the process will go to step 407: determine whether the current time is later than the reminder terminating time.

If the measurement remains unfinished after the lower reminder canceling time range limit 75, the controlling module 50 will determine whether the current time is later than the reminder terminating time 76. If it is determined that the current time is later than the reminder terminating time 76, step 410 will be performed; otherwise, the process will go to step 408. In this embodiment, the controlling module 50 knows the current time through the clock device 40 and determines whether the current time is later than 10:00 AM. If it is determined that the current time is later than the reminder terminating time 76, that is, 10:00 AM, the standby state of the reminding device 30 will be canceled; otherwise, the process will go to step 408.

If it is determined that the current time is not later than the reminder terminating time, then the process will go to step 408: start the reminding device.

If it is determined that the current time is not later than the reminder terminating time 76, the controlling module 50 will start the reminding device 30. In this embodiment, the controlling module 50 starts the audio reminding device for generating sound to remind the user 80. In addition to starting the reminding device 30 in the human physiological signal measuring apparatus 1, the controlling module 50 is capable of sending a signal to the electronic device 90 to start a reminding mechanism on the electronic device 90 directly, for example, generating preset sound. Furthermore, in a multiple-user mode, it is feasible to start a reminding mechanism on different electronic devices used by different users, so as to remind the users separately.

Step 409: wait for a fixed time period.

Once the reminding device 30 starts, it will wait for a fixed time period before performing step 406 and step 407. In this embodiment, the audio reminding device generates sound once every one minute. During the aforesaid time period, the reminding device 30 performs step 406 and step 407 again to determine whether the measurement is done and determine whether the current time is later than the reminder terminating time 76. If the user 80 has not finished the measurement, steps 406-409 will be repeatedly performed before the reminder terminating time 76, that is, 10:00 AM. Once the user 80 finishes the measurement or once it is determined that the current time is later than 10:00 AM, the standby state of the audio reminding device will be canceled.

Step 410: cancel the standby state of the reminding device.

If the user 80 has finished the measurement task, then the standby state of the reminding device 30 can be canceled. Alternatively, if the current time is later than the reminder terminating time 76, the standby state of the reminding device 30 will be canceled even though the user 80 has not completed the measurement task.

Upon completion of the aforesaid steps of determining whether to release the reminding device, the steps below are performed (on the presumption that the user 80 has finished the measurement).

Step 501: record measurement time and numerical data.

Upon completion of the measurement, the human physiological signal measuring apparatus 1 of the present invention further records a measurement-related time 71 and numerical data of the physiological signal 70. Referring to FIG. 6, in this embodiment, the physiological signal 70 is a blood pressure-related signal, and thus the human physiological signal measuring apparatus 1 records a blood pressure level 79 of the user 80, and the measurement-related time 71, which is the time taken to measure the physiological signal 70, is also recorded. A point to note is that the human physiological signal measuring apparatus 1 of the present invention is not limited to a blood pressure-related signal, but may include any other physiological signals, such as an electrocardiogram-related signal or a blood sugar-related signal, nor is the measurement-related time 71 limited to the time taken to measure the physiological signal 70.

Step 502: send to the external electronic device.

Upon completion of the recording operation, the record is sent to the external electronic device 90, such that the user 80 can read the record thereon, though the disclosure of the present invention is not limited thereto; instead, the user 80 can also read the record directly with the displaying device 65.

In conclusion, the features of the present invention are completely different from those of the prior art in terms of objectives, means, and effects. However, it should be noted that the above embodiments are illustrative of the principle and effect of the present invention only, and they should not be interpreted as restrictive of the scope of the present invention. Hence, persons skilled in the art can make modifications and changes to the aforesaid embodiments without going against the technical principle and spirit of the present invention. Accordingly, the extent of legal protection for the rights claimable toward the present invention should be defined by the appended claims.

What is claimed is:

1. A method for measuring a human physiological signal, the method being for use with a human physiological signal measuring apparatus, the apparatus comprising a reminding device, the method comprising the steps of:
receiving a reminder canceling time range;
measuring a physiological signal with the human physiological signal measuring apparatus; and
determining whether a measurement-related time for measuring the physiological signal falls within the reminder canceling time range, followed by releasing the reminding device in response to an affirmative determination.

2. The method of claim 1, wherein the reminder canceling time range is 15 minutes to 90 minutes.

3. The method of claim 1, further comprising the step of receiving a preset reminder starting time and obtaining the reminder canceling time range based on the preset reminder starting time, wherein the preset reminder starting time falls within the reminder canceling time range.

4. The method of claims 1, further comprising the step of receiving an identification data and identifying a user identity based on the identification data.

5. The method of claims 1, wherein the physiological signal is one of a blood pressure-related signal, an electrocardiogram-related signal, or a blood sugar-related signal.

6. The method of claims 3, wherein the reminder canceling time range and the preset reminder starting time are set by an external electronic device or directly by the human physiological signal measuring apparatus.

7. The method of claims 1, wherein the measurement-related time is a time taken to measure the physiological signal.

8. The method of claims 1, wherein the reminding device is an audio reminding device.

9. An apparatus for measuring a human physiological signal, the apparatus comprising:
a receiving module for receiving a reminder canceling time range;
a measuring module for measuring a physiological signal;
a reminding device for reminding a user to measure the physiological signal;
a clock device for providing a measurement-related time for measuring the physiological signal;
a memory for storing the reminder canceling time range;
a controlling module electrically connected with and adapted to control the receiving module, the measuring module, the reminding device, the clock device, and the memory for determining whether the measurement-related time falls within the reminder canceling time range and releasing the reminding device in response to an affirmative determination.

10. The apparatus of claim 9, wherein the reminder canceling time range is 15 minutes to 90 minutes.

11. The apparatus of claim 9, wherein the receiving module receives a preset reminder starting time and obtains the reminder canceling time range based on the preset reminder starting time, wherein the preset reminder starting time falls within the reminder canceling time range.

12. The apparatus of claims 9, wherein the receiving module receives an identification data and identifies a user identity based on the identification data.

13. The apparatus of claims 9, wherein the physiological signal is one of a blood pressure-related signal, an electrocardiogram-related signal, and a blood sugar-related signal.

14. The apparatus of claims 11, further comprising:
a time setting device for setting the reminder canceling time range or the preset reminder starting time; and
a displaying device for displaying a measurement record.

15. The apparatus of claims 11, wherein the reminder canceling time range and the preset reminder starting time are set by an external electronic device.

16. The apparatus of claims 9, wherein the measurement-related time is a time taken to measure the physiological signal.

17. The apparatus of claims 9, wherein the reminding device is an audio reminding device.
